(19) United States
(12) Patent Application Publication
(10) Pub. No.: US 2021/0260094 A1
(43) Pub. Date: Aug. 26, 2021

(54) Polylactose, a prebiotic dietary fiber

(71) Applicant: Regents of the University of Minnesota, Minneapolis, MN (US)

(72) Inventors: Tonya C. Schoenfuss, Buffalo, MN (US); Daniel D. Gallaher, Roseville, MN (US); Ryan Fink, Minneapolis, MN (US)

(21) Appl. No.: 17/254,033

(22) PCT Filed: Jun. 28, 2019
(86) PCT No.: PCT/US19/39875

§ 371 Date: Dec. 18, 2020

Related U.S. Application Data
(60) Provisional application No. 62/692,206, filed on Jun. 29, 2018.

(51) Int. Cl. A61K 31/715; A23L 33/125

(52) U.S. Cl. CPC A61K 31/715; A23L 33/125

(57) ABSTRACT

Polylactose is used as a prebiotic either as a supplement or in human food. The polylactose is essentially free from deleterious sugar caramelization by-products such as 5-hydroxymethyl furfural.

### FIELD

The present disclosure relates to prebiotics and in particular it relates to polylactose as a prebiotic.

### BACKGROUND

Prebiotic food ingredients confer physiological benefits by modulation of gut microbiota, which leads to benefits to the host such as improved immune function, and the alleviation of metabolic derangements due to disease states such as obesity and type-2 diabetes. The most widely used prebiotics in the United States are inulin and oligofructose.

Prebiotic oligosaccharides are ingredients that remain intact during their transit through the stomach and the small intestine, for example such ingredients are not broken down or altered by stomach acid or digestive enzymes, and upon reaching the large intestine promote conditions beneficial to the consumer. One effect is a change in the ratio of the colon microbiota which results in benefits to host health (Gibson and Roberfroid, 1995). The health benefits that have been attributed to this stimulation of desirable organisms in the colon include an improvement in immune system functioning (Mussatto and Mancilha, 2007), modulation of lipid metabolism due to fermentation products, the synthesis of vitamins, and the inhibition of growth of harmful bacteria by a change in fecal pH (Gibson and Roberfroid, 1995; Playne and Crittenden, 2004). Certain gut microbial populations are also thought to modulate adverse conditions such as inflammation that are associated with obesity and type 2 diabetes (Roberfroid et al., 2010).

The generally accepted prebiotic food ingredients are all in the category of dietary fiber. The Codex Committee on Nutrition and Foods for Special Dietary Uses recommends that dietary fiber be defined to include carbohydrates with a degree of polymerization (DP)>10, that are not hydrolyzed by enzymes in the small intestine of humans (Codex Alimentarius Commission, 2008). By DP is meant the number of monomeric units or repeat units in a macromolecule or polymer or oligomer. The committee also comments that decisions to include carbohydrates with 3-9 DP should reside with national labeling authorities. Many authorities include DP 3-9 and most agree with Codex that when a carbohydrate is derived from a raw food material by physical, enzymatic or chemical means, or has been synthetically produced, it should have a demonstrated physiological beneficial effect in humans (Codex Alimentarius Commission, 2008; de Menezes et al., 2013). At the Food and Agriculture Organization of the United Nations (FAO) technical meeting on prebiotics, a prebiotic was defined as a “non-viable food component that confers a health benefit on the host associated with modulation of the microbiota”, and it was recommended that a prebiotic should show both bifidogenic activity, and a health benefit to the host (AGNS and FAO, 2007; Pineiro et al., 2008). It was also stated that a prebiotic can be a fiber, but that a fiber is not necessarily a prebiotic. Because of the benefits of soluble dietary fiber, the prebiotic ones in particular, the soluble food fiber market is predicted to grow annually by 10% from S165 million in 2010 to S321 million by 2017 (Frost and Sullivan, 2012). Future forecasts for the use of prebiotics such as inulin, oligofructose and galacto-oligosaccharides (GOS) illustrate the need to ensure that an economical oligosaccharide production process is in place in order to meet the future demand

Commonly used prebiotic fibers in the food industry in the U.S. include inulin, fructooligosacharides (FOS), polydextrose, galacto-oligosaccharides (GOS), and resistant starch, that is, starch that is resistant to digestion. The evaluation of these ingredients for their prebiotic activity is varied, as the “beneficial effects” that are desired are varied. The ability of the organisms to alter the microbiome is one measure that is well established to be a component of determining if something is a prebiotic. In the past, it was very difficult to understand the diversity and relative numbers of organisms present because the majority were unculturable. But with the rapid advance of genomic testing, and the dramatic reduction in costs per sample, understanding how gut microorganisms are affected by the ingestion of fibers can be easily accomplished. The most extensively evaluated group of prebiotics, inulin and related fructooligosaccharides, have been shown in many studies to alter gut microbiota to the benefit of the host when compared to a control (Cani, et al., 2007; Dewulf, et al., 2012; Raninen, et al, 2011).

Inulin has a varied chain length, with a DP that varies from 3-60 (Raninen et al, 2011). Fructooligosaccharides, which may be derived from inulin, can be produced in varying DPs. The length of the fructan affects the speed and location of fermentation with shorter DPs being generally more fermentable (Hernot, et al., 2009). Polydextrose, on the other hand, has an average DP of 12 but is highly branched, has varied glycosidic bonds between sugars, and contains a range of DPs up to approximately 30 sugars (Burdock and Flamm, 1999). The comparative effects of consuming inulin and polydextrose on blood glucose and lipids, bowel function, fermentability and location of fermentation by colon microbiota were summarized in the review by Raninen et al. in 2011. It was reported that inulin is fermented to a larger degree than polydextrose and its fermentation occurs in the proximal colon, whereas polydextrose is mainly fermented in the distal colon (Raninen et al., 2011). Polydextrose was also found to be less fermentable than shorter chain lengths of FOS (but not longer chains), by measuring gas production and short-chain fatty acid production in vitro human fecal microflora experiments (Hernot et al., 2009). In studies conducted with humans, polydextrose was demonstrated to alter the Bateroidetes:Firmicutes ratio by increasing the abundance of Bacteroidetes by 12% in fecal samples over a control group that did not receive fiber (Holscher et al., 2015). Castabile et al. (2011) also reported beneficial changes in fecal microorganisms in human subjects consuming polydextrose with increases in microorganisms that produce butyrate, as well as reduced genotoxicity of fecal water, potentially indicating reduction in risk factors for colon cancer. Lamichhane et al. (2014) have also reported that the consumption of polydextrose led to increased levels of acetate and propionate, and increased Bifidobacterium in feces. Given that polydextrose is less fermentable than shorter chain-length FOS, it is understandable that its tolerance is higher since it produces less gas and approximately 30-50% is excreted (Auerbach et al., 2007). Up to 90 g/day or 50 g as a single dose is the expected mean laxative threshold for polydextrose, whereas the threshold for inulin and FOS ranges from 20-30 g/day (Flood, et al., 2004).

Polydextrose is produced by polymerizing glucose or maltose with an acid catalyst (typically citric or phosphoric acid) and sugar alcohol (sorbitol) to create branched oligomers. Polydextrose is used in food as a bulking agent and is considered a prebiotic soluble fiber. Industrial production is via a batch process, under vacuum, and the sorbitol is added as a way to quench the reaction (Rennhard, 1973). There are also products that are hydrogenated to convert end-groups to sorbitol to make them less reactive (non-reducing) (Burdock and Flamm, 1999). Due to its acidic nature, it is also sometimes neutralized with KOH or other bases (Burdock and Flamm, 1999). Similar to other heated sugars, it contains 5-hydroxymethylfurfural (HMF); however, its concentration must not exceed 0.1% per Codex Alimentarius (FAO and WHO, 2015). A promising method of fiber production by a continuous process via extrusion was reported in the late 1990's. Hwang et al. published two reports on the extrusion of glucose, as well as lactose, to produce oligosaccharides and achieved polymerization yields of 94 and 46%, respectively (Hwang et al., 1997 & 1998).

Other food ingredients that seem to have ability to reduce body fat and fatty liver, and improve blood glucose control are viscous dietary fibers, such as guar gum and hydroxypropyl methylcellulose. However, viscous dietary fibers are difficult to incorporate into foods in a way that is acceptable to most consumers, as they generally have an objectionable mouthfeel; that is, they are very slimy and gummy.

### SUMMARY

One aspect of this disclosure comprises a method of using polylactose as a prebiotic either as a supplement or incorporated into human food.

Another aspect of this disclosure comprises a method for producing polylactose while reducing the concentration of 5-hydroxymethyl furfural. A further aspect includes the use of a continuous extrusion process to produce the polylactose while reducing the concentration of hydroxymethyl furfural. In a further aspect, dietary fiber concentration is increased while sugar concentration is decreased.

In yet another aspect of this disclosure, a method comprises utilizing polylactose to reduce inflammation. In another aspect metabolic endotoxemia is reduced through the use of polylactose. In another aspect, insulin sensitivity is increased and blood glucose control is improved. In another aspect tendency to adiposity is reduced. In yet another aspect, fatty liver condition is reduced in dietary induced obesity (DIO).

In yet another aspect, liver cholesterol is reduced.

In yet another aspect, the use of polylactose increases the amount of beneficial gut microbiota in the DIO animal model.

In yet another aspect of this disclosure the amount of fiber that is fermented between the different forms of fiber is evaluated.

### BRIEF DESCRIPTION OF THE DRAWINGS

FIG. 1 is a photographic view comparing color of polylactose before (left) and after (right) mixed bed carbon filtration.

FIG. 2 is a graphical view illustrating body weights of rats among dietary groups.

FIG. 3 is a graphical view illustrating daily energy intake of rats among dietary groups.

FIG. 4 is a graphical view illustrating cecal content pH of rats among dietary groups.

FIG. 5 is a graphical view illustrating cecum weight of rats among dietary groups.

FIG. 6 is a graphical view illustrating blood glucose response of rats among dietary groups.

FIG. 7 is a graphical view illustrating blood glucose response of rats among dietary groups.

FIG. 8 is a graphical view illustrating epididymal fat pad weight of rats among dietary groups.

FIG. 9 is a graphical view illustrating plasma leptin concentration of rats among dietary groups

FIG. 10 is a graphical view illustrating effect of polylactose on liver lipid concentration of rats among dietary groups

FIG. 11 is a schematic view illustrating beta-diversity of large intestinal microbial populations among animals.

FIG. 12 is a graphical view illustrating the abundance of several species of bacteria that are considered beneficial for health

FIG. 13 is a graphical view illustrating the amount of cholesterol in the liver in relation to various prebiotics.

FIG. 14 is a graphical view illustrating abundance of Bifidobacterium resulting from polylactose in comparison to other prebiotics.

FIG. 15 is a graphical view illustrating abundance of Lactobacillus resulting from polylactose in comparison to other prebiotics.

FIG. 16 is a graphical view illustrating abundance of Akkermansia muciniphila resulting from polylactose in comparison to other prebiotics.

FIG. 17 is a graphical view illustrating the ratio of Firmicutes:Bacteroidetes in relation to various prebiotics.

### DETAILED DESCRIPTION

This describes the use of polylactose as a prebiotic for humans. Polylactose as described herein can be used as either a supplement or in human foods as a prebiotic. A prebiotic is, by definition, a substance, usually a dietary fiber, that alters the colonic microflora in a positive way and imparts a health benefit on the host.

An animal study was conducted in which polylactose as well as the two best established prebiotic dietary fibers, polydextrose and fructooligosaccharides, were fed to rats. Not all of the results have been tabulated and realized as of the filing date of this disclosure.

Polydextrose, fructooligosaccharides and polylactose were fed as part of a high fat diet to induce a mild obesity, an impaired blood glucose control, and a fatty liver. The results are set forth below. The findings, thus far, demonstrate that polylactose is highly fermentable, as measured by the drop in cecal pH (the cecum being the first part of the large intestine in rats) and an increase in cecum weight.

Polylactose was found to be more fermentable than both polydextrose and fructooligosaccharides. The greater fermentation of polylactose suggests it will have a more profound effect on the colonic microflora. In terms of health benefits to the host, polylactose significantly reduced body fat, as measured by a lighter epididymal fat pad, when compared to the high fat control diet. In rodents, fat tissue is present, in part, as discrete tissues, referred to as fat pads. Changes in fat pad weight reflect changes in whole body fat. In addition, plasma leptin concentrations showed a trend towards a lower concentration when compared to the high fat diet control. Plasma leptin concentration is proportional to total body fat.

Glycemic control was also examined Rats fed polylactose tended to have better blood glucose control than rats fed the high fat control diet. In addition, rats fed polylactose had a lesser degree of fatty liver compared to rats fed the high fat control diet. Fatty liver is a precursor to non-alcoholic fatty liver disease (NAFLD), a condition prevalent among obese individuals and diabetics, and can lead to liver failure.

Thus, based on the findings to date as exemplified in the examples set forth below, polylactose appears to be an extraordinarily effective prebiotic, far more effective than the prebiotics such as polydextrose and fructooligosaccharides which currently are considered to be the best prebiotics.

In addition, this disclosure describes a method of producing a high purity polylactose suitable for use as a prebiotic. The polylactose produced is a nearly tasteless powder suitable for use as a supplement or for incorporation into a very large number of foods.

The method comprises using a continuous extrusion process to produce polylactose, a mixture of oligosaccharides, from lactose, glucose and citric acid. The polylactose was produced via a condensation reaction of sugars with an acid catalyst,

Along with other caramelization products, HMF is formed in sugars heated with acids. HMF has shown toxicity in in vitro and animal studies. Thus, reducing its formation through optimum processing conditions and/or sample treatments is beneficial. However, the concentration of HMF in the final, ground product has at no time been measured above 0.1%, which is the specification limit of polydextrose (WHO, 1998) HMF content was measured in the extrudates by RP-HPLC/UV. Extrudates produced by a lower feed rate resulted in significantly higher levels of HMF and were also darker. Sample treatments to remove HMF using Amberlite (an ion exchange resin) and activated charcoal were evaluated. Filtration over Amberlite lead to a 27-57% reduction in HMF. Preliminary results indicate that charcoal treatment resulted in even further HMF reduction (>95%), such that the concentration of HMF in the polylactose preparation was <0.05%. Both treatments also resulted in products of lighter color, thus caramelization products other than HMF were also removed from the product. Additionally, charcoal and Amberlite treatments removed remaining citric acid catalyst from about 2% to about 0.05%. Both Amberlite and charcoal are available in food grade options, and both methods can be upscaled. Moreover, there are further chromatography media used to decolorize sugars and juices commercially available that may allow for further HMF reduction. Additionally, use of chromatography and filtration media can also be used to remove residual lactose. During manufacturing of GOS, only about 30-50% of the lactose is converted to GOS by the enzymatic reaction, so the solution was purified to increase the fiber content and reduce lactose. Prior art product for sale on the market contains from to 7%-33% lactose (Clasado, 2014: Friesland Foods Domo, 2008; GTC Nutrition, 2009; Yakult, 2010). These differences are entirely based on post-manufacturing processing by techniques such as carbon filtration, membrane filtration, and ion exchange chromatography as described in their GRAS submissions. The same procedures have been found to be suitable for polylactose to reduce the lactose concentration.

The HMF in the method of this disclosure was almost entirely removed in a purification step (filtration with activated charcoal and a mixed-bed ionic resin containing activated charcoal and Amberlite). In other words, the product was essentially free of HMF. By essentially free is meant that the product contained less than 0.0009% HMF by weight, preferably less than 0.1% HMF by weight and most preferably less than 0 005 HMF by weight.

When determining whether an ingredient or food has prebiotic activity, both in vitro and in vivo methods are recommended by the FAO (Pineiro et al., 2008). They recommend that bifidogenic effects are not enough, and that physiologic health benefits should be demonstrated. Examples of physiological benefits that could be measured include satiety, endocrine function changes that affect food intake and energy usage, absorption of nutrients, improved immune function, improved blood lipid levels, changes in bowel function, and markers for cancer risk (Pineiro et al., 2008).

The diet-induced obesity (DIO) animal model provides an excellent model to examine the effect of prebiotics and soluble fibers on a number of important health benefits simultaneously. In this model, rats are fed a high fat diet for 10-14 weeks, which results in the development of obesity, insulin resistance, a worsening of glucose control, a low grade inflammatory state, due at least in part to metabolic endotoxemia, fatty liver, and a reduction in Bifidobacterium (Cani et al., 2007; Brockman et al., 2014). Incorporation of the prebiotic oligofructose into a high fat diet has been shown to reduce adiposity, improve glucose control, reduce inflammatory cytokines, decrease plasma lipopolysaccharide (LPS, a marker of endotoxemia) and increase cecal bifidobacteria (Cani et al., 2007). We have found that incorporation of the soluble fiber guar gum into a high fat diet similarly reduces adiposity, improves insulin sensitivity and glucose control, eliminates fatty liver, and alters energy metabolism to a more normal state (Brockman et al., 2014). Consistent with these animal studies, feeding prebiotic dietary fibers, such as oligofructose, to obese people increases the number of bifidobacteria and decreases serum LPS concentrations (Dewulf et al., 2012), strongly suggesting that the DIO animal model is relevant to the human situation.

Polylactose was found to be non-digestible in vitro (Tremaine et al., 2014) and therefore is a dietary fiber.

To produce the polylactose of this disclosure the amounts of lactose, glucose and citric acid can be varied, to optimize the yield and reduce the amount of brown color. The amounts were varied from about 2 to 6% citric acid, about 20% glucose was used, and the remainder was lactose. The addition of glucose allows the sugars to melt more quickly in the extruder, resulting in better fiber yields, since the glucose has more water associated with it and its melting temperature is also lower.

### Example 1

Production of Polylactose by Twin-Screw Extrusion while Removing Hydroxymethylfurfural for the Feeding Trials

Polylactose was produced from 74% lactose (Refined edible fine grind lactose, >99% purity, Davisco Foods International, Inc., Eden Prairie, Minn., USA), 20% glucose (dextrose monohydrate, Roquette America, Inc., Geneva, Ill., USA) and 4% citric acid, anhydrous (Jungbunzlauer, Basel, Switzerland). The dry material was blended on an IMS-1 ribbon blender (Bepex International LLC, Minneapolis, Minn., USA) in a forward and reverse direction for 2 minutes each. The blend was then placed in the hopper of a K-Tron Soder K-ML-KT20 loss-in-weight feeder (K-Tron Ltd., Niederlenz, Switzerland) for feeding into the Bühler 44 mm co-rotating twin-screw extruder DNDL 44, which has a length to diameter ratio of 28 (Bühler AG, Uzwil, Switzerland). The feed rate was 15 kg/hour. Thirty-nine screw elements of varying pitch angles were used, including 9 kneading block elements (6 forward and 3 reverse), 20 forward conveying elements and 10 reverse conveying elements. The design included many reverse elements in order to maximize time that the product spent in the extruder to achieve greater reaction time, as well as more efficient conversion of mechanical energy to heat which resulted in higher temperatures. The barrel zones had different heating temperatures as described from the feeder inlet to outlet: barrel zones #2 and #3 were set at 238° C., zone #4 was set at 238° C., zones #5 and #6 were set at 238° C. and there was no heating in zone #7. A heat transfer control system model H47212DT (Mokon, Buffalo, N.Y., USA) was used to maintain the temperature for each barrel's oil heating jacket. No die plate was used at the outlet of the extruder. After cooling and hardening, the glassy material was ground in a hammer mill (Fitzpatrick, Waterloo, Canada) to a fine powder. The polylactose was solubilized in water and the polylactose was partially purified by filtration in a mixed-bed column that was composed of a glass column (1558 cm3) that was filled with 15 g of diatomaceous earth and then 400 g of Cabot NORIT GAC 1240 PLUS granular activated carbon was added on top. An ion exchange resin consisting of 50 g of Amberlite FPA 53 OH— and 50 g of Ambersep 200 H+(Megazyme International) was added between the diatomaceous earth and the activated carbon layers. The column was first rinsed with 3000 mL of reverse osmosis water, and then 800 mL of a 200 mg/mL solution of polylactose solubilized in water was added to the column with 200 mL of reverse osmosis water. 1000 mL of reverse osmosis water was used to rinse the column. All samples eluted at a rate of approximately 3 mL/min. The purified polylactose samples were spray dried on an APV Anhydro Type I spray dryer (SPX FLOW, Inc., Charlotte, N.C., USA) with an APV CF-100 atomizer (SPX FLOW, Inc.). The spray drying conditions were inlet temperature, 185° C.; outlet temperature, 90° C.; flow rate, 220 mL/min; atomizer, 24,000 rpm. Eluent, after purification, and spray dried samples were analyzed for dietary fiber by the integrated dietary fiber method (item number K-INTDF 02/15, Megazyme International). This method is based on AOAC Method 2009.01 with minor alterations. D-ribose (Sigma Aldrich) was used as the internal standard for high performance liquid chromatography (HPLC) analysis. The HPLC used was a Beckman (Beckman Coulter Inc., Fullerton, Calif.) consisting of a System Gold 508 auto sampler, System Gold 125 solvent module pump, and a programmable CO20 column heater (Torrey Pines Scientific, Carlsbad, Calif.). A Transgenomics CH0-411 column (Omaha, Nebr., USA) was used for separation, and a Sedex 85 LT low temperature evaporative light scattering detector (ELSD-LT) (Shimadzu Corporation) was used to detect peaks. The HPLC conditions used were a column temperature of 80° C., flow rate of 0.3 mL/min and a double distilled water mobile phase. The ELSD nebulizer temperature was set at 40° C. and the nitrogen pressure was 250 kPa.). Lactose was determined using an enzymatic test kit (Megazyme Lactose/Sucrose/D-Glucose Kit, Megazyme, Bray, Ireland). Hydroxymethyl furfural was quantified using a method adapted from Truzzi et al., 2012. Polylactose samples were diluted in double distilled water to a concentration of 100 mg/mL and filtered through a 0.45 μm syringe filter. The sample was injected into a Shimadzu LC-2010 HT system with a UV-Vis detector (Shimadzu Corporation). All HPLC analyses used an YMC Pack ODS-AM column (YMC CO. Ltd., Kyoto, Japan). The conditions used were an isocratic mobile phase, water-methanol (95+5 v/v); flow rate, 0.8 mL/min; injection volume, 20 μL; column temperature 30° C.; detection, 285 nm. The standard curve for polylactose HMF quantification (R²=0.998) used 5 standard solutions of HMF in double distilled water (0.05 mg/mL, 0.075 mg/mL, 0.1 mg/mL, 0.15 mg/mL and 0.2 mg/mL) and the standard curve filtered polylactose HMF quantification (R²=0.998) also used 5 HMF standards (9.38×10-5 mg/mL, 1.88×10-4 mg/mL, 3.75×10-4 mg/mL, 7.50×10-4 mg/mL and 1.50×10-3 mg/mL). Citric acid was determined using the citric acid (citrate) manual assay procedure (item number K-CITR 11/14, Megazyme International). A 53 Shimadzu UV-1800 spectrophotometer was used for measuring absorbance as per the method (Shimadzu Corporation, Kyoto, Japan).

As shown in Table 1, mixed-bed carbon filtration of the unfiltered polylactose increased the low molecular weight soluble dietary fiber (LMWSDF; essentially polylactose), lactose, and glucose concentration from the starting material (labeled polylactose unfiltered), but reduced the citric acid concentration.

TABLE 1
Compositional analysis of polylactose and polylactose that was treated with a mixed
bed filtration method¹

| | LMWSDF | | | Citric Acid |
|---|---|---|---|---|
| Sample | (%)² | Lactose (%) | Glucose (%) | (%) |
| Polylactose unfiltered | 35.75 ± 0.93^{a} | 16.63 ± 0.92^{a} | 5.82 ± 0.48^{a} | 2.02 ± 0.17^{a} |
| Mixed Bed Carbon Filtered | | | | |
| Polylactose | 50.13 ± 1.16^{b} | 21.92 ± 0.66^{b} | 6.27 ± 0.22^{a} | 0.05 ± 006^{b} |
| (Large Scale Benchtop Method) | | | | |

¹Values are means ± one standard deviation (N = 4),
^{a-c}Means without a common superscript letter within the same column are significantly different (p < 0.05)
²Low molecular weight soluble dietary fiber

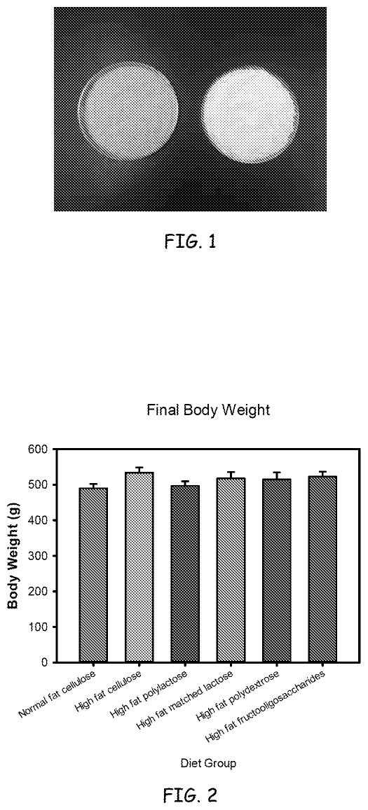

The heat and pressure of extrusion, the process used to produce polylactose, also produces Maillard browning and caramelization products. One of these is 5-hydroxymethyl-2-furalaldehyde, commonly known as hydroxymethyl furfural (HMF). HMF has been associated with adverse health effects for humans, including genotoxicity, upper respiratory tract irritation and skin irritation (European Food Safety Authority, 2011; Morales, 2009). Mixed bed carbon filtration reduced the hydroxymethyl furfural (HMF) content of polylactose by over 99.5% and reduced the absorbance at 420 nm by over 77% (Table 2), resulting in a white powder (FIG. 1).

FIG. 1. Color comparison of polylactose before (left) and after (right) mixed bed carbon filtration

TABLE 2
Effect of mixed bed carbon filtration and spray drying on polylactose color¹

| | | Abs (420 | | | |
|---|---|---|---|---|---|
| Sample | HMF (%)² | nm) | L | a | b |
| Polylactose unfiltered | 0.090 ± 0.005^{a} | 0.363 ± 0.013^{a} | 86.17 ± 0.68^{a} | −7.13 ± 0.13^{a} | 22.10 ± 0.17^{a} |
| Mixed Bed Carbon | 0.000355 ± 0.00^{b} | 0.077 ± 0.003^{b} | 93.25 ± 0.15^{b} | −5.66 ± 0.03^{b} | 7.01 ± 0.11^{b} |
| Filtered Polylactose | | | | | |
| Spray Dried Polylactose | | | | | |

¹Values are means ± standard deviation (N = 4),
^{a-c}Means without a common superscript letter within the same column are significantly different (p < 0.05).
²Hydroxymethylfurfural

The final product was thus a white material that contained approximately 50% polylactose, about 22% free lactose, and was essentially free of HMF. This material was deemed quite appropriate for the animal trial as set forth in Example 2.

### Example 2

Evaluation of the Impact of Polylactose on Biochemical Markers of Inflammation, Metabolic Endotoxemia, Glucose Control, Insulin Sensitivity, Adiposity, and Fatty Liver in a Diet-Induced Obesity (DIO) Animal Model

72 male Wistar rats (Charles River of Wilmington, Mass.) were fed normal fat (NF) or high fat (HF, 50% fat by kcal) diets ad libitum containing various fibers (6% fiber of interest and 3% cellulose, by weight); including cellulose (NFC and HFC), polylactose (HFPL), matched lactose (HFML, matched to the residual lactose in the HFPL diet), and two established prebiotic fibers, polydextrose (HFPD) and fructooligosaccharides (HFFOS) for 10 weeks.

| | Normal | | High fat | High fat | High fat | High fat |
|---|---|---|---|---|---|---|
| | fat | High fat | poly- | matched | poly- | oligo- |
| Component | cellulose | cellulose | lactose | lactose | dextrose | fructose |
| Cellulose | 90.00 | 90.00 | 30.00 | 90.00 | 30.00 | 30.00 |
| Polylactose | 0.00 | 0.00 | 119.69 | 0.00 | 0.00 | 0.00 |
| Polydextrose | 0.00 | 0.00 | 0.00 | 0.00 | 60.00 | 0.00 |
| Lactose | 0.00 | 0.00 | 0.00 | 0.00 | 0.00 | 0.00 |
| Oligofructose | 0.00 | 0.00 | 0.00 | 0.00 | 0.00 | 60.00 |
| Lard | 86.66 | 200.00 | 200.00 | 200.00 | 200.00 | 200.00 |
| Soybean Oil | 28.89 | 66.66 | 66.66 | 66.66 | 66.66 | 66.66 |
| Sucrose | 100.00 | 50.00 | 50.00 | 50.00 | 50.00 | 50.00 |
| Maltodextrin | 132.00 | 132.00 | 132.00 | 132.00 | 132.00 | 132.00 |
| Cornstarch | 337.45 | 202.47 | 202.47 | 202.47 | 202.47 | 202.47 |
| Casein | 200.00 | 200.00 | 200.00 | 200.00 | 200.00 | 200.00 |
| Mineral Mix | 35.00 | 35.00 | 35.00 | 35.00 | 35.00 | 35.00 |
| Vitamin Mix | 10.00 | 10.00 | 10.00 | 10.00 | 10.00 | 10.00 |
| L-Cystine | 3.00 | 3.00 | 3.00 | 3.00 | 3.00 | 3.00 |
| Choline Bitr. | 2.50 | 2.50 | 2.50 | 2.50 | 2.50 | 2.50 |
| TBHQ | 0.01 | 0.01 | 0.01 | 0.01 | 0.01 | 0.01 |
| TOTAL (g) | 1025.51 | 991.64 | 1051.33 | 991.64 | 991.64 | 991.64 |
| Kcal/g diet | 4.03 | 4.79 | 4.69 | 4.79 | 4.79 | 4.79 |

Diet composition, percent by weight

| | Normal | | High fat | High fat | High fat | High fat |
|---|---|---|---|---|---|---|
| | fat | High fat | poly- | matched | poly- | oligo- |
| Component | cellulose | cellulose | lactose | lactose | dextrose | fructose |
| % CHO | 56.95 | 38.45 | 35.86 | 38.45 | 38.45 | 38.45 |
| % Protein | 20.30 | 20.30 | 20.30 | 20.30 | 20.30 | 20.30 |
| % Fat | 11.56 | 26.67 | 26.67 | 26.67 | 26.67 | 26.67 |
| % Fiber | 9.00 | 9.00 | 8.56 | 9.00 | 9.00 | 9.00 |

During weeks 8 and 9 of feeding the experimental diets, rats underwent oral glucose, insulin (by intraperitoneal (i.p.) injections), and pyruvate (by i.p. injections) tolerance tests. At the end of week 10, blood was collected, centrifuged, and plasma collected and stored at −80° C. The liver (to examine fatty liver) and epididymal fat pads (to determine adiposity) were excised, weighed, and flash frozen in liquid nitrogen and stored at −80° C.

Plasma leptin concentration was assessed as an additional indicator of body fat accumulation.

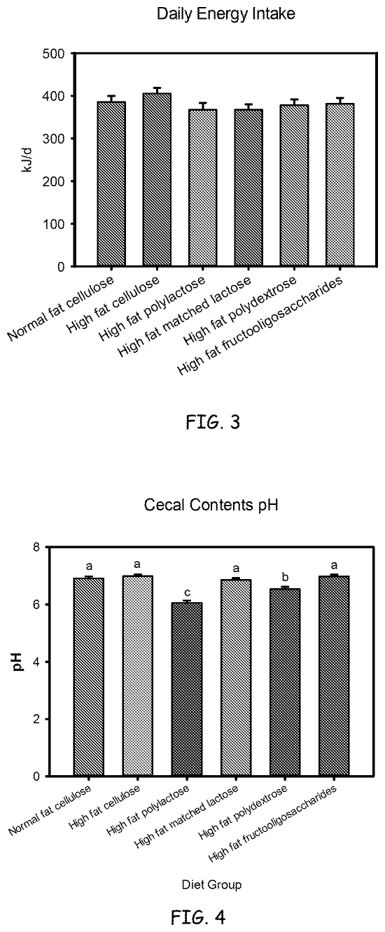

Final body weights and daily energy intake of the rats did not differ among the dietary groups, as shown in FIGS. 2 and 3.

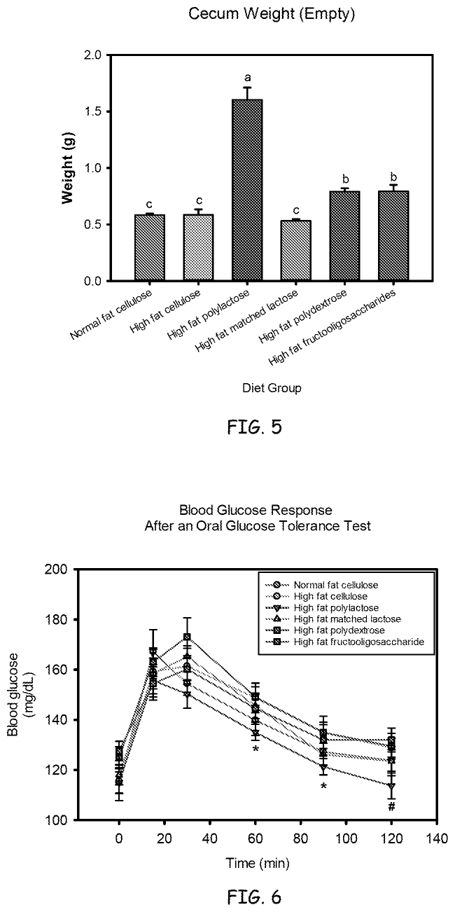

In order to assess the degree of fermentation of the polylactose, polydextrose, and fructooligosaccharides, cecal pH and cecal tissue weight were determined. The cecum is the first part of the large intestine in the rat, and is the site of greatest fermentation. With greater fermentation, pH drops and cecal tissue weight increases as illustrated in FIGS. 4 and 5

Cecal pH was significantly reduced in the high fat (HF) polylactose group and the HF polydextrose group, relative to the HF cellulose control group. However, the reduction in cecal pH was greater with polylactose. Cecal weight (empty of contents) was dramatically increased in the HF polylactose group, relative to the HF cellulose control group, and somewhat increased by the HF polydextrose and HF fructooligosaccharides groups. These findings indicate that polylactose is stimulating more fermentation than polydextrose or fructooligosaccharides at the same dietary concentration of 6% of the diet.

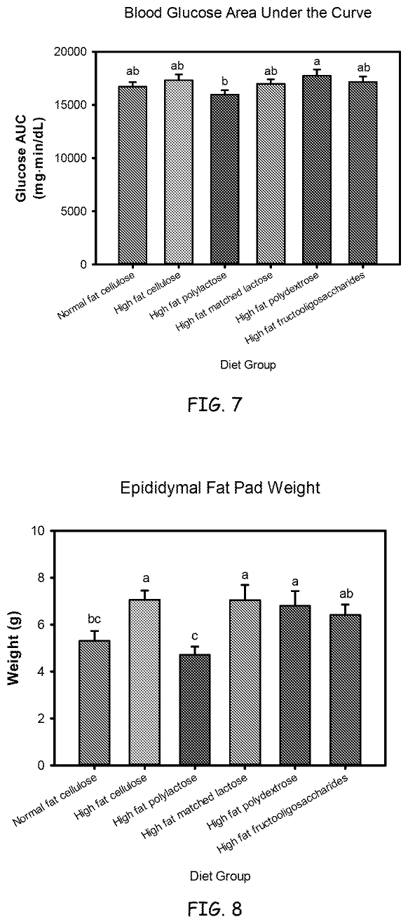

Blood glucose control was examined by an oral glucose tolerance test, as illustrated in FIGS. 6 and 7.

The blood glucose response to an oral load of glucose shows that at 60, 90, and 120 minutes, animals fed the HF polylactose diet had the lowest blood glucose concentrations. Integrated over the 120 minutes (the area under the curve, AUC), there was a trend for a reduction in glucose AUC in the HF polylactose group. By Student's t-test, the difference between the HF cellulose control group and the HF polylactose group was close to statistical significance (p=0.056).

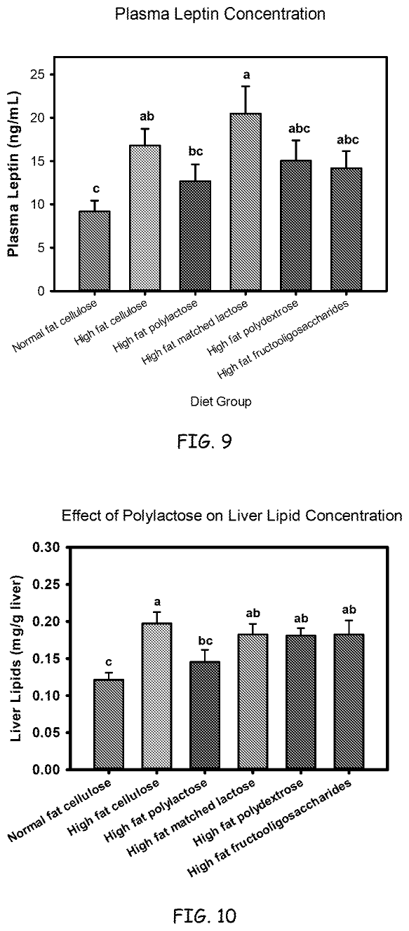

The effect of the diets on body fat was assessed by measuring the weight of the epididymal fat pad and by measuring plasma leptin. Fat pad weight reflects whole body fat, as does plasma leptin, a hormone released from fat tissue (an adipokine) as shown in FIGS. 8 and 9.

Animals fed the HF polylactose diet had the lightest epididymal fat pad weight, even numerical lighter than the normal fat cellulose control as shown in FIG. 10. Thus, polylactose appeared to dramatically reduce adiposity compared to polydextrose or fructooligosaccharides. Plasma leptin concentration in the HF polylactose group was decreased from the HF cellulose group, but this difference was not statistically significant.

### Example 3

Evaluation of the Impact of Polylactose on the Microbiota in a DIO Animal Model

Total DNA was extracted from cecal contents samples using the QIAamp DNA Stool Mini Kit (Qiagen, Carlsbad Calif.). DNA corresponding to the V5 and V6 hypervariable regions of the full-length 16S rDNA was amplified by PCR using primers previously described (Wang et al., 2007; Lazarevic et al., 2009). The taxonomy of microorganisms assigned to the V6 hypervariable regions is very similar to the taxonomy assigned to microorganisms obtained via analysis of full-length SSU rRNA (Huse et al., 2008), making this a cost effective approach to obtain near complete taxonomic information (Lazarevic et al., 2009). The PCR primers contain a unique sequence tag (Binladen, 2007), such that amplicons from each sample contain a unique identifier sequence. The amplicons from each of the samples were pooled together and sequenced on an Illumina/Solexa Sequencer at the University of Minnesota Genomics Center facility at the University of Minnesota. Since each amplicon is identified by a different primer tag, it will allow us to deconvolute sequence data arising from a single sequence run. Sequence data was obtained by the paired-end read method. Using this approach, about 30 million reads were obtained from each sequence run, and resulted in the collection of approximately 681,000 million reads of taxonomically useful 16S rDNA from each sample.

Fatty liver is a precursor to non-alcoholic fatty liver disease (NAFLD), a condition common in obese and diabetic individuals. NAFLD can progress to liver cirrhosis and liver necrosis, and is therefore considered a serious health issue. The fat concentration of the livers was determined in order to examine the effect of the diets on fatty liver.

As can be seen, animals fed the HF polylactose diet had less fat in their livers compared to the HF cellulose control group, and did not differ from the normal fat cellulose control group. There was no significant reduction in liver fat by polydextrose or fructooligos accharides.

Prebiotics by definition must cause a change in the microflora of the large intestine in a way that is beneficial to the host.

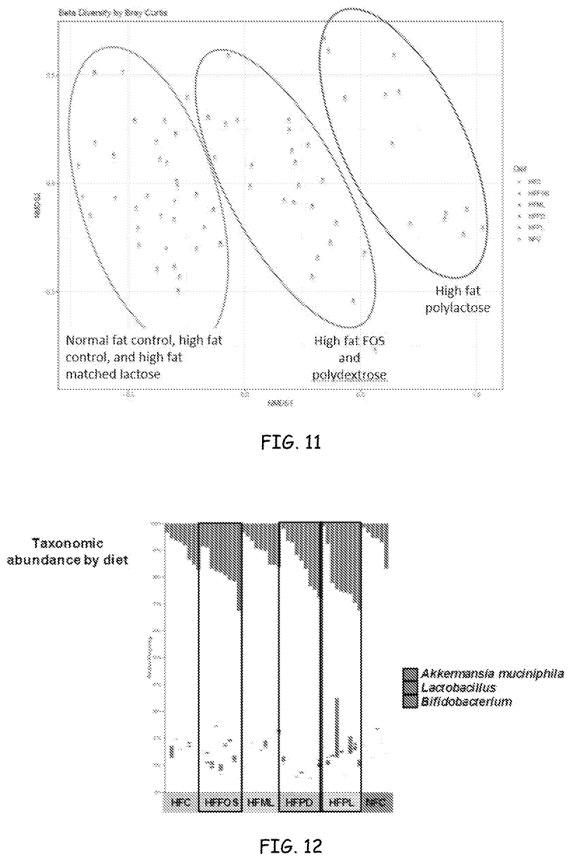

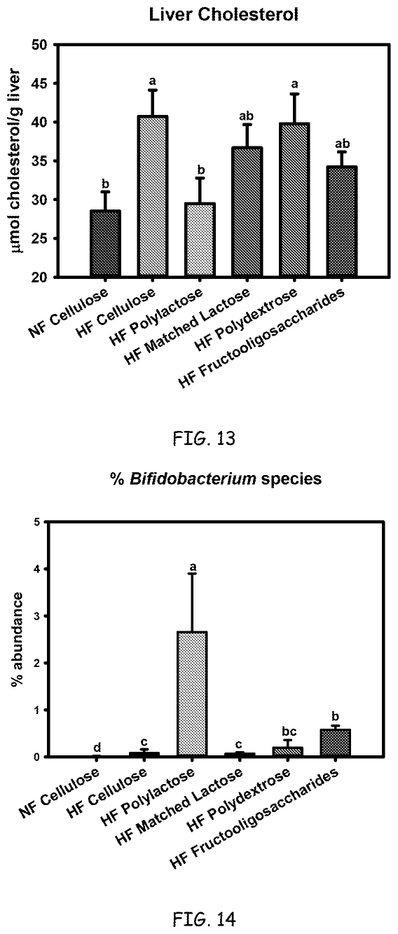

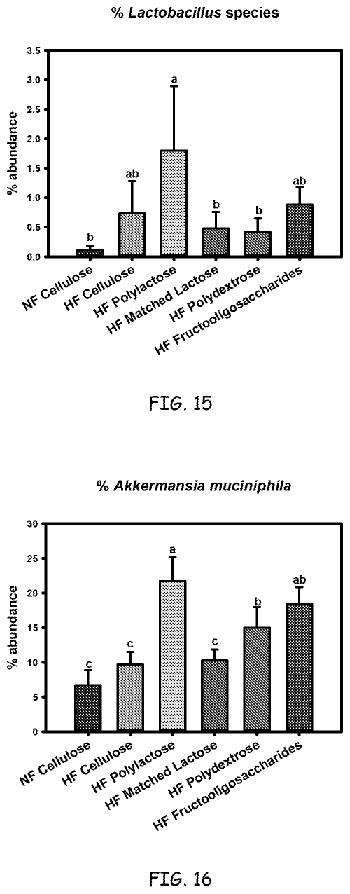

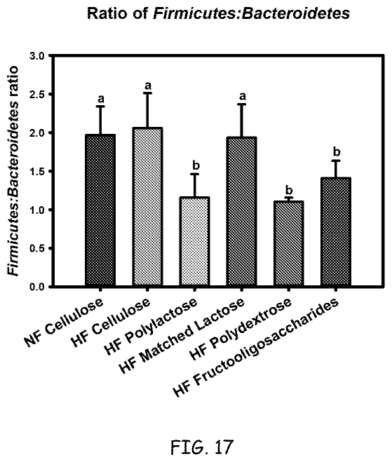

Beta-diversity was examined Beta-diversity indicates how the large intestinal microbial populations differ among animals. In the plots of FIG. 11, each point represents an individual animal.

Beta-Diversity as a Bray Curtis PCoA plot is shown in FIG. 11.

The different dietary groups are separated into three distinct groupings by the beta-diversity plot. This indicates that the microbial populations in the normal fat cellulose (normal fat control), HF cellulose (high fat control), and HF matched lactose are similar to each other, the HF polydextrose and HF fructooligosaccharides (FOS) groups are similar to each other, and that HF polylactose is producing a unique profile.

The abundance of several species of bacteria that are considered beneficial for health are shown in FIG. 12.

Most notably, there is a much greater abundance of Bifidobacterium in the HF polylactose group (HFPL) than either the HF polydextrose or HF fructooligosaccharide groups. Whether differences exist in the abundance of Lactobacillus among the groups is less apparent. The abundance of Akkermansia muciniphila appears greater in HF polylactose, HF polydextrose, and HF fructooligosaccharide groups relative to all other groups.

The microbiome analysis completed to date is strongly suggestive that polylactose alters the microbiome in a way that would be beneficial to the host.

### Example 4 (Prophetic Example)

Evaluation of the Amount of Fiber that is Fermented Between the Different Forms of Fiber Evaluated

To determine the degree to which the test oligosaccharide fibers are fermented in the large intestine, the soluble dietary fiber in the fecal material will be determined, using a procedure that measures non-digestible oligosaccharides such as polylactose, fructooligosaccharides, and polydextrose, the integrated dietary fiber assay AOAC method 2009.01 (AOAC, 2009). Thus, the difference between intake and excretion of the oligosaccharides represents their fermentation.

What is claimed is:

1. A method comprising using polylactose as a prebiotic in humans.

2. The method of claim 1 wherein the polylactose is essentially free from deleterious caramelization by-products.

3. The method of claim 2 wherein the caramelized by-products comprise 5-hydroxymethylfurfural.

4. The method of claim 1 wherein the polylactose is used as a supplement or incorporated in a food.

5. The method of claim 1 wherein the effect of polylactose comprises a reduction in inflammation.

6. The method of claim 1 wherein the effect of polylactose comprises a reduction of metabolic endotoxemia.

7. The method of claim 1 wherein the effect of polylactose comprises an increase in insulin sensitivity and blood glucose control is improved.

8. The method of claim 1 wherein the effect of polylactose comprises a reduction in adiposity.

9. The method of claim 1 wherein the effect of polylactose comprises a reduction in fatty liver condition in dietary induced obesity.

10. The method of claim 1 wherein the effect of polylactose comprises an increase in the amount of beneficial gut microbiota.

11. A method of producing polylactose essentially free from caramelized sugars, the method comprising:
extruding a mixture of lactose, glucose and citric acid through an extruder producing a polylactose mixture;
purifying the polylactose mixture through an ion exchange column thereby essentially eliminating the concentration of caramelized sugars to make a polylactose essentially free from caramelized sugars; and
producing a powder from the polylactose mixture.

12. The method of claim 11 wherein the caramelized sugars include 5-hydroxymethylfurfural.

13. The method of claim 11 and further comprising filtering the polylactose through activated charcoal.

14. The method of claim 11 and further processing the polylactose through chromatography.
